(19) United States
(12) Patent Application Publication
(10) Pub. No.: US 2002/0096181 A1
(43) Pub. Date: Jul. 25, 2002

(54) Method for performing a hysterectomy

(76) Inventor: Darius Samimi, M.D., Corona Del Mar, CA (US)

Correspondence Address: RICHARD E. BEE, P.O. BOX 10544, COSTA MESA, CA, 92627, US

(21) Appl. No.: 09/766,084

(22) Filed: Jan. 19, 2001

(51) Int. Cl. A61B 19/00

(52) U.S. Cl. 128/898

(57) ABSTRACT

An improved method for performing a hysterectomy wherein the cardinal ligaments and the uterosacral ligaments attached to a uterus are not severed. Also, the wall of the vaginal apex is not cut. This is accomplished by coring through the cervical stroma of the uterus close to the wall of the endocervical canal and transformation zone and removing the endocervical canal and transformation zone from the cervical stroma. The opening left in the cervical stroma after removal of the endocervical canal and transformation zone is closed with sutures. This technique is practically bloodless. The nerve plexuses and the support system of the female internal organs are preserved. The chance of future cervical cancer is substantially eliminated. This is truly a technique for the 21^{st} century.

### DESCRIPTION

1. Technical Field

This invention relates to surgical methods for performing hysterectomies on female patients.

2. Background of the Invention

A hysterectomy involves the removal of the uterus from the abdomen of a female patient. The traditional method of performing a hysterectomy is to sever the uterosacral ligaments, the cardinal ligaments and the uterine vessels attached to the uterus before entering the vaginal fornix. The uterus is then severed from the vagina in a circular fashion at the cervico-vaginal junction. To access this area, the bladder is pushed down and, if necessary, dissected free of any attachments to the uterus.

This traditional procedure causes significant damage to the nerves in the Frankenhauser nerve plexus, the vesical nerve plexus and various regional nerves such as the nerves to the clitoris, the urethra and the vestibular bulbs. This traditional procedure also causes a major impairment of the pelvic support system for the vagina and other major complications.

### SUMMARY OF THE INVENTION

The present invention provides an improved surgical method of hysterectomy for removing a uterus including its endocervical canal and transformation zone in a manner which preserves important nerve entities and pelvic support structures while, at the same time, reducing the risk of cervical cancer. This improved method includes coring through the cervical stroma of a uterus close to the wall of the endocervical canal and transformation zone so as to leave the bulk of the cervical stroma in tact and connected to the cardinal ligaments, the uterosacral ligaments and the wall of the vaginal apex. The endocervical canal and transformation zone are removed from the cervical stroma and the opening left in the cervical stroma by the removal of the endocervical canal and transformation zone is closed with sutures. This new technique is a relatively bloodless technique.

For a better understanding of the present invention, together with other and further advantages and features thereof, reference is made to the following description taken in connection with the accompanying drawings, the scope of the invention being pointed out in the appended claims.

### BRIEF DESCRIPTION OF THE DRAWINGS

Referring to the drawings:

FIG. 1 is a longitudinal cross-sectional view of a human female uterus;

FIG. 2 is a transverse cross-sectional view of the FIG. 1 uterus taken along section line 2-2 of FIG. 1;

FIG. 3 is a transverse cross-sectional view of the FIG. 1 uterus taken along section line 3-3 of FIG. 1;

FIG. 4 is longitudinal cross-sectional view of the portion of the FIG. 1 uterus that is removed from the abdomen of the patient;

FIG. 5 is a perspective view showing the portion of the cervix that remains in the body of the patient after removal of the portion of the uterus shown in FIG. 4; and

FIG. 6 is a perspective view of the cervix portion of FIG. 5 after closure with sutures of the opening left in the cervix by removal of the endocervical canal and transformation zone.

### DETAILED DESCRIPTION OF THE ILLUSTRATED EMBODIMENT

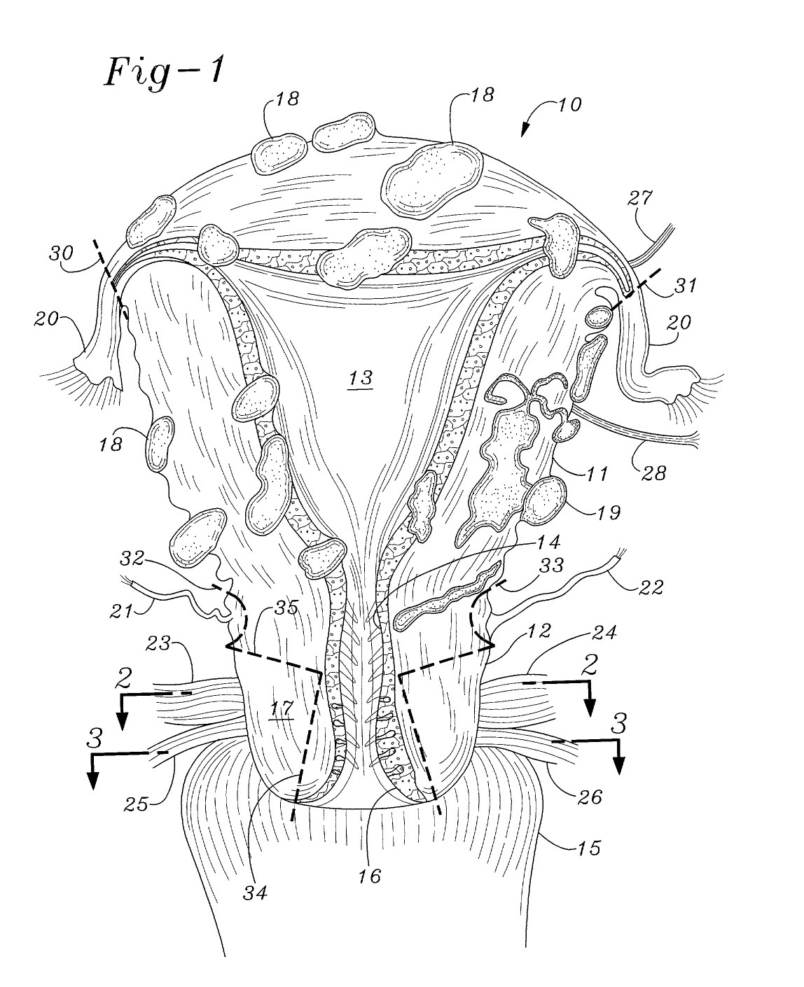

Referring to FIG. 1 of the drawings, there is shown a longitudinal cross-sectional view of a human female uterus 10. The upper portion 11 of uterus 10 is called the corpus and the lower portion 12 is called the cervix. A typical uterus in a non-pregnant adult female human being is approximately three inches in length and has a width of approximately two inches at its widest. The diameter of the cervix 12 is a little less than one inch along section line 2-2. The larger cavity 13 in the upper region of uterus 10 is called the endometrial cavity. A tubular passageway 14, called the endocervical canal, runs from the lower end of endometrial cavity 13 to the top end or apex of a vagina 15. Cervix 12 extends a short distance into the vagina 15, the upper end of vagina 15 being attached to and closed by the lower portion of cervix 12. An outwardly flaring portion 16 at the lower end of the endocervical canal 14 is called the transformation zone or T-zone. The fibrous tissue 17 forming the interior of the cervix 12 and surrounding the endocervical canal 14 is called cervical stroma.

As an example of a problem, the roundish blotches 18 shown on uterus 10 are fibroid tumors which sometimes form on the wall of a uterus. Sometimes, such as in the case of fibroid 19, they extend outwardly a noticeable distance from the uterus. For present purposes, it is assumed that some of the fibroids on uterus 10 are harmful and that this is the reason for removing the uterus 10.

Attached to the upper sides of uterus 10 are a pair of Fallopian tubes 20 which extend to different ones of the two ovaries (not shown). It is through one of the Fallopian tubes 20 that the egg enters the interior of the uterus 10. Attached to the wall of uterus 10 near the upper end thereof are ovarian ligaments 27 and round ligaments 28. Closer to cervix 12 are two uterine arteries 21 and 22. A short distance downwardly are two sets of cardinal ligaments 23 and 24 which are attached to the two sides of the cervix 12. These ligaments extend outwardly and their far ends are attached to the pelvic wall (not shown). Cardinal ligaments 23 and 24 provide a significant amount of support for uterus 10. Immediately below the cardinal ligaments are two sets of uterosacral ligaments 25 and 26 which are attached to the two sides of cervix 12 and extend outwardly to and are attached to the pelvic wall (not shown).

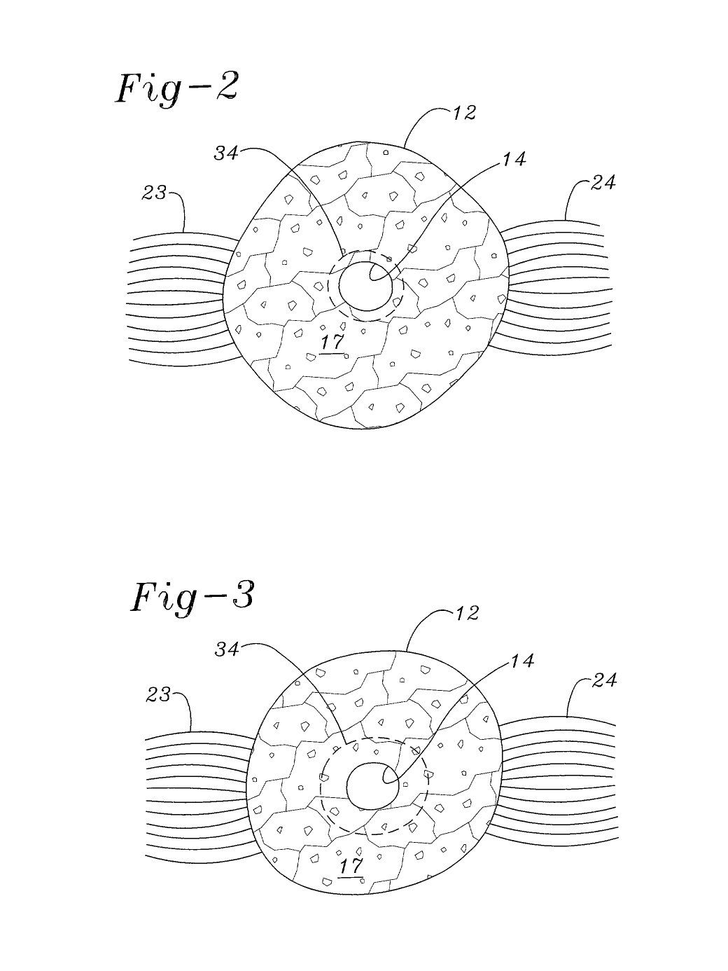

FIGS. 2 and 3 are transverse cross-sections of cervix 12 of FIG. 1, taken along section lines 2-2 and 3-3, respectively. Among other things, these figures show the circular natures of cervix 12 and endocervical canal 14.

The present invention provides an improved surgical method for removing a uterus with reduced trauma to the patient and fewer postoperative problems. It is, of course, necessary to make various cuts and incisions in order to free the uterus from the body of the patient. The lines along which cuts are made are indicated by the broken lines in FIGS. 1-3. As seen in FIG. 1, the Fallopian tubes 20 and ovarian ligaments 27 are severed, as indicated at 30 and 31. Round ligaments 28 are severed. The uterine arteries 21 and 22 are clamped and their ends are cut out of the cervix 12, as indicated by cut lines 32 and 33.

Next comes the severing of the uterus 10 from the vagina 15. The present invention does this in a special way. In particular, a circular incision 34 is made into the cervical stroma 17 close to the wall of the endocervical canal 14 and transformation zone 16 so as to form a severed core which includes the endocervical canal 14 and transformation zone 16. The circular nature of this incision 34 is seen in FIGS. 2 and 3. This incision 34 extends from the lower end of cervix 12 upwardly to a level a short distance above the cardinal ligaments 23 and 24. A lateral incision 35 is made into the side of the cervix 12 to a depth sufficient to join with the longitudinal coring incision 34. Lateral incision 35 is made completely around the cervix 12 so as to separate the upper portion 17a of cervical stroma 17 from the lower portion 17b of cervical stroma 17.

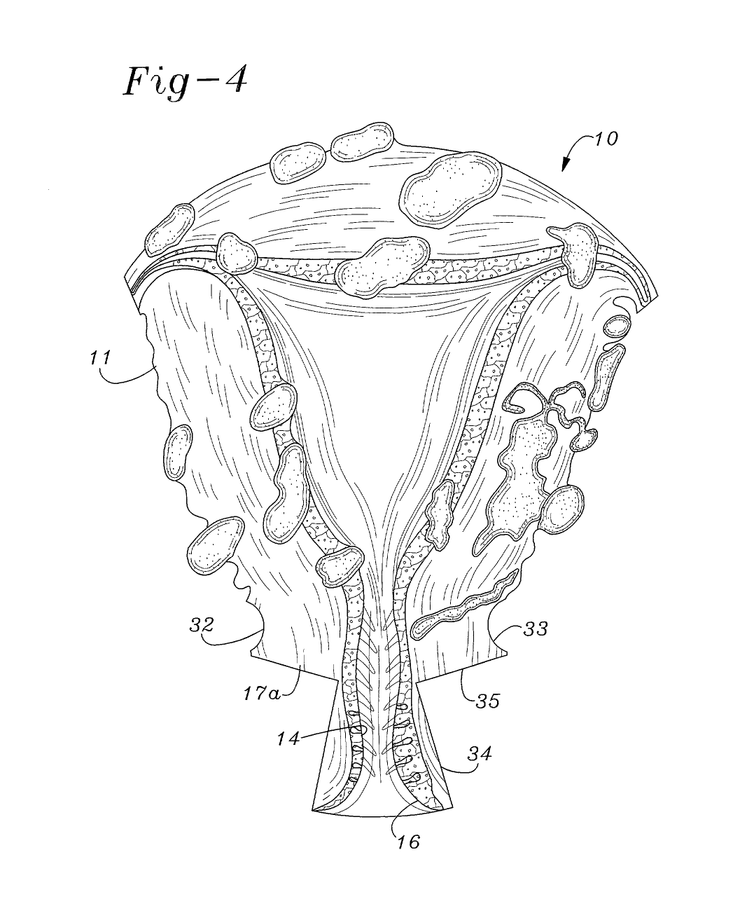

FIG. 4 shows the severed uterus 10 after removal from the body of the patient. The results of coring incision 34 and lateral incision 35 are clearly seen in FIG. 4. As shown, the endocervical canal 14 and transformation zone 16 are included with the part of the uterus 10 which is removed.

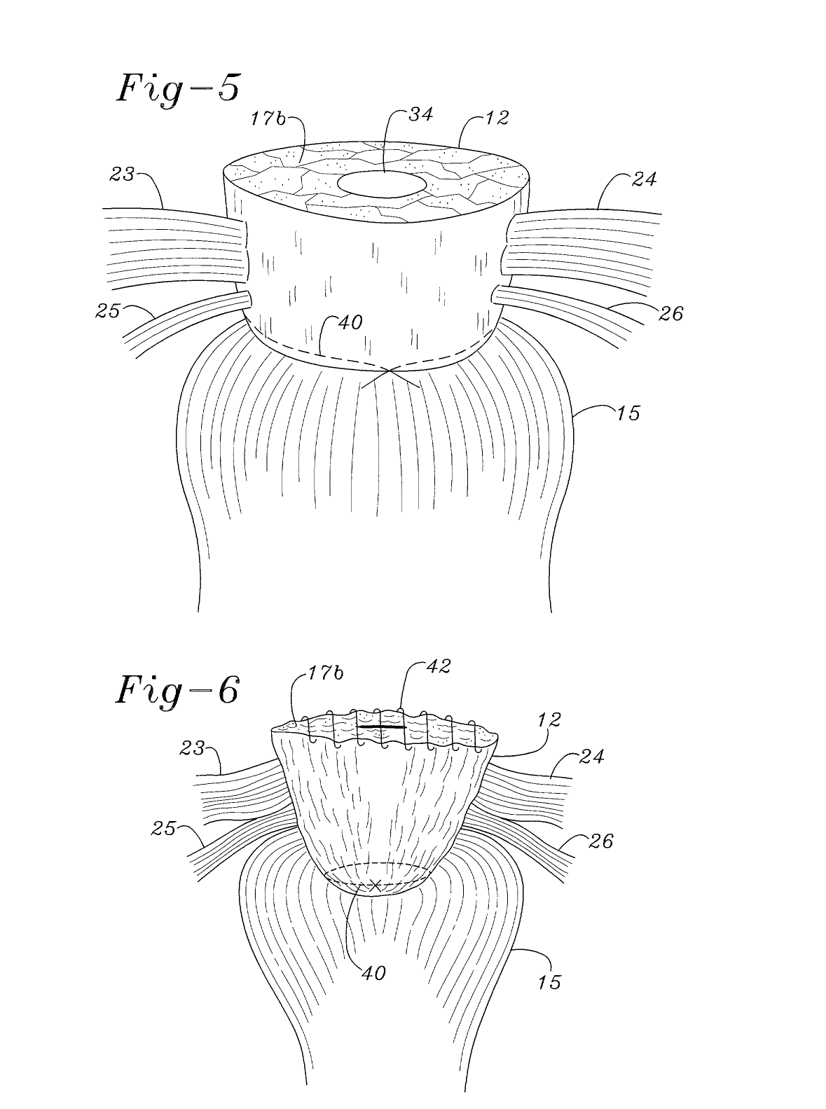

FIG. 5 shows the portion of cervix 12 that remains in the body of the patient. It is a doughnut shaped structure that remains attached to the cardinal ligaments 23,24, the uterosacral ligaments 25,26 and the wall of the apex or fornix of vagina 15. Thus, the procedure of the present invention leaves in tact the bulk of the lower portion 17b of cervical stroma 17.

FIG. 6 shows the cervix portion of FIG. 5 after closure with sutures of the opening or passageway 34 left in the cervix 12 by removal of the core portion containing the endocervical canal 14 and transformation zone 16. As seen in FIG. 5, a first suture 40 is circumferentially attached to the lower end of cervix 12 so as to encircle the lower opening of cervix 12. This is accomplished by weaving suture 40 in and out of the outer wall of cervix 12. This is preferably done before the coring incision 34 is made, the free ends of suture 40 being left untied. After completion of the coring procedure and after removal of the core containing endocervical canal 14 and transformation zone 16, the free ends of suture 40 are pulled tight so as to force a closure of the lower opening in cervix 12. The ends of suture 40 are then tied together to make the closure permanent.

A second suture 42 is used to close the upper opening in the cervical stroma portion 17b. Sutures 40 and 42 are preferably of the delayed absorption type. After a short period of time, the squeezed together wall portions of passageway 34 will be permanently interconnected by fibrous tissue growth.

As seen in FIGS. 1 and 3, the core formed by coring incision 34 has a slight conical shape to it, the diameter of the core becoming smaller as one moves in the upward direction. As mentioned, the coring incision 34 is made close to the wall of the endocervical canal 14 and transformation zone 16 so as to leave the bulk of lower cervical stroma portion 17b in tact. The lateral distance between the upper end of core incision 34 and the wall of endocervical canal 14 is typically about one-quarter of an inch. Incision 34 runs downwardly pretty much in a straight line to the bottom of cervix 12 at a point about one-quarter of an inch laterally of the mouth of transformation zone 16.

Core incision 34 may be made in either the downward or the upward direction. In the downward case, the lateral incision 35 is made, after which the cutting instrument is turned in the downward direction to make core incision 34. In the upward case, the cutting instrument is inserted into the vagina 15 and moved in the upward direction to make core incision 34. The cutting instrument may be, for example, either an electrosurgical Bovie knife or, alternatively, a laser knife.

With the method of the present invention, neither the cardinal ligaments nor the uterosacral ligaments are severed. This avoids injury to the Frankenhauser nerve plexus and to the vesical nerve plexus, as well as injury to other nerves such as nerves to the clitoris, urethra and vestibular bulbs. The present invention also preserves the pelvic support system for the vagina. This minimizes postoperative problems related to bladder dysfunction, as well as possible vaginal sexual dysfunction. Furthermore, removal of the endocervical canal and transformation zone greatly reduces the risk of developing a cervical cancer. At the same time, applicant's technique is one of the best bloodless techniques of modern medicine.

While there has been described what is at present considered to be a preferred embodiment of this invention, it will be obvious to those skilled in the art that various changes and modifications may be made therein without departing from the invention and it is, therefore, intended to cover all such changes and modifications as come within the true spirit and scope of the invention.

What is claimed is:

1. A surgical method of hysterectomy for removing a uterus including its endocervical canal and transformation zone in a manner which preserves important nerve entities and pelvic support structures, while at the same time reducing the risk of cervical cancer, such method comprising:
coring through the cervical stroma of a uterus close to the wall of the endocervical canal and transformation zone so as to leave the bulk of the cervical stroma in tact and connected to the cardinal ligaments, the uterosacral ligaments and the wall of the vaginal apex;
removing the endocervical canal and transformation zone from the cervical stroma;
and closing with sutures the opening left in the cervical stroma after removal of the endocervical canal and transformation zone.

2. A surgical method of hysterectomy for removing a uterus including its endocervical canal and transformation zone in a manner which preserves important nerve entities and pelvic support structures, while at the same time reducing the risk of cervical cancer, such method comprising:
cutting through the cervical stroma of a uterus above the cardinal ligaments so as to separate the upper portion of the cervical stroma from the lower portion of the cervical stroma;
coring through the lower portion of the cervical stroma close to the wall of the endocervical canal and transformation zone so as to leave the bulk of the lower portion of the cervical stroma in tact and connected to the cardinal ligaments, the uterosacral ligaments and the wall of the vaginal apex;
removing the endocervical canal and transformation zone from the cervical stroma, thereby eliminating the chance of ureteral injuries which is a major complication of traditional techniques;
and closing with sutures the opening left in the lower portion of the cervical stroma after removal of the endocervical canal and transformation zone, thereby reducing the chance of excessive blood loss which is a major problem with traditional techniques.

3. A surgical method of hysterectomy for removing a uterus including its endocervical canal and transformation zone in a manner which preserves important nerve entities and pelvic support structures, while at the same time reducing the risk of cervical cancer, such method comprising:
circumferentially attaching a suture to the cervical stroma of a uterus so as to encircle the lower opening of the cervix of the uterus;
coring through the cervical stroma of the uterus close to the wall of the endocervical canal and transformation zone so as to leave the bulk of the cervical stroma in tact and connected to the cardinal ligaments, the uterosacral ligaments and the wall of the vaginal apex;
removing the endocervical canal and transformation zone from the cervical stroma;
drawing tight and tying together the ends of the circumferential suture;
and closing with a second suture the upper opening left in the cervical stroma after removal of the endocervical canal and transformation zone.
